(19) United States
(12) Patent Application Publication
(10) Pub. No.: US 2015/0127114 A1
(43) Pub. Date: May 7, 2015

(54) BIONIC JOINT DEVICE WITH CLOSED LUBRICATION

(71) Applicant: Dezheng Zhao, Wulumuqi (CN)

(72) Inventor: Dezheng Zhao, Wulumuqi (CN)

(21) Appl. No.: 14/117,455

(22) PCT Filed: Feb. 27, 2012
(86) PCT No.: PCT/CN12/71646

§ 371 Date: Apr. 29, 2014

(30) Foreign Application Priority Data
May 17, 2011 (CN) 201120156371.0

(51) Int. Cl. A61F 2/30; A61F 2/38; A61F 2/32

(52) U.S. Cl. CPC A61F 2/30; A61F 2/32; A61F 2/30756; A61F 2/3836; A61F 2002/30663; A61F 2002/30673; A61F 2002/307; A61F 2002/30563; A61F 2002/30589
USPC 623/22.13; 623/23.41

(57) ABSTRACT

A bionic joint device with closed lubrication comprises a main body bionic bone (1), a driven bionic bone (4), an elastic easily-stretching cavity housing (2), a cavity sealing hoop (3), an elastic shock-proof material (5), a high-strength wear-resistant self-lubricating material (6) and a powerful inelastic limit connection (7). The elastic easily-stretching cavity housing (2) is internally filled with lubricating grease (8). The device is applicable to the robotics field and the artificial joint field.

TECHNICAL FIELD

The present invention provides an innovative joint device applicable to the fields of robots and artificial joints, especially a bionic joint device with closed lubrication that is capable of multi-directional bending and twisting and position-limiting control. By varying its shape and size, the bionic joint device with closed lubrication can be used in different parts of the human body and in animals.

### BACKGROUND ART

Most of the prior-art bionic joints perform position-limiting control by means of metal hinges and various complex screws and pins, and place rigorous requirements on size and shape due to their complex mechanical structure, so they can hardly be used for the human body or as joint structures varying in size. In addition, they do not really simulate the shape of the human skeleton. These deficiencies have restricted the applications for mechanical joints of robots and for bionic joints. In addition, prior-art bionic joint research focuses on ways of changing the hinges and the control, so that the bionic degree is very small. In particular, the popularization and development of prior-art bionic joints are restricted due to the deficiency of lubrication in the artificial bionic joint field.

### CONTENT OF THE INVENTION

The present invention is intended to provide a bionic joint device with closed lubrication, which can bend and twist in multiple directions by simulating the articular tissues of animals and can limit and control these motions. This bionic joint device can be fully applied to humanoid robots or other quadruped machinery, thus enabling them to act more flexibly and freely. If bionic materials such as high-quality, oil-proof silicone rubber is used in the medical bionic joint field, a lubricating grease that is harmless to humans can be used for these artificial human joints, so as to effectively lubricate the artificial joints and successfully popularize artificial joints. In addition, the present invention makes it cost-effective and easy to produce artificial limbs.

The object of the present invention is achieved by: A bionic joint device with closed lubrication, comprising a master bionic bone 1, a slave bionic bone 4, an elastic cavity housing 2, a cavity sealing clamp 3, an elastic shock-absorbing material 5, a high-strength, wear-resistant, self-lubricating material 6, and a powerful inelastic limit connection 7, characterized in that: the elastic cavity housing 2 is filled with a lubricating grease 8 that satisfies the lubrication requirements of the high-strength, wear-resistant self-lubricating material 6; the lubricating grease 8 maintains the lubrication action between the master bionic bone 1 and the slave bionic bone 4; the elastic cavity housing 2 and the lubricating grease 8 protect the high-strength, wear-resistant, self-lubricating material 6 and ensure good bending, twisting, and sliding performance of the joint device; the elastic cavity housing 2 maintains lubrication action and prevents leakage of lubricating grease in the process of bending and stretching; the cavity sealing clamp 3 seals the elastic cavity housing 2 by means of the clamping slots on the master bionic bone 1 and the slave bionic bone 4; and glue is applied on the contact surfaces to prevent leakage of the lubricating grease 8 inside the elastic cavity housing 2. With variations in shape and size, the bionic joint device can be applied for different parts of the human body and in animals.

The elastic shock-absorbing material 5 is used to mitigate external impacts and vibrations; the high-strength, wear-resistant, self-lubricating material 6 is used to reduce the friction force between the contact surfaces and to prolong their useful life; the powerful inelastic limit connection 7 can be placed at multiple positions along the circumference as needed according to the bending direction with a view to limiting the bending degree; and the powerful inelastic limit connection 7 controls the gap size between the master bionic bone 1 and the slave bionic bone 4. For example, the limit connection can be slightly extended in the front of the joint to control the angle of bending, and provides limit protection as well as safety protection and prevents the occurrence of disjointing if it is placed on two sides. The bending direction of the joint can also be changed by changing the shapes of the contacting surfaces; for example, a knee joint that has two supporting points and can bend forwards and backwards, or a spherical hip joint that provides a rotation function. The tensile deformation of the elastic cavity housing can prevent leakage of lubricating grease contained inside the cavity and ensure a good lubrication effect of the wear-resistant material between the master bone and the slave bone. The shock-absorbing elastic material can satisfactorily mitigate and buffer various vibrations, keep the joint in good condition, and reduce impacts on other organs.

The bionic joint device with closed lubrication can be used as a spherical, rotatable hip joint, which is capable of rotating and moving multi-directionally; the bionic joint is pressed tightly and sealed by means of a threaded connection 9, thereby ensuring a good sealing effect of the cavity.

The present invention works on the following principle: The bionic joint in each closed, lubricating, stretchable and elastic protection cavity can bend, twist, and slide as directed by externally acting forces, has a basic structure completely similar to human joints, can bend, twist, and slide like human joints, and can simulate joints in different parts of the human body and animals by varying its size and shape; therefore, such a bionic joint can be applied in the technical fields of humanoid robots and quadruped machinery. The present invention can simulate diversified functions for example, bending, stretching out and pulling back, twisting, and limiting of animal joints and can be fully applied in the technical fields of humanoid robots or quadruped machinery, thereby enabling humanoid robots or quadruped machinery to act more flexibly and freely. With advances in material science, the present invention can also be used as substitution joints in the body in the field of medical artificial bionic joints.

### DESCRIPTION OF FIGURES

FIG. 1 shows a sectional view of the bionic joint device with closed lubrication provided by the present invention.

FIG. 2 shows the joint wrapping state for the elastic cavity of the bionic joint device provided by the present invention.

FIG. 3 shows a sectional view of a knee joint provided by the present invention, which has two contact points and can bend forwards and backwards.

FIG. 4 shows a sectional view of a spherical, rotatable hip joint provided by the present invention.

FIG. 5 shows a sectional view of the pulled-back state. In this figure, note the location of the inelastic limit connection along the circumference according to bending needs.

FIG. 6 shows a top view of the pulled-back state.

### PREFERRED EMBODIMENTS

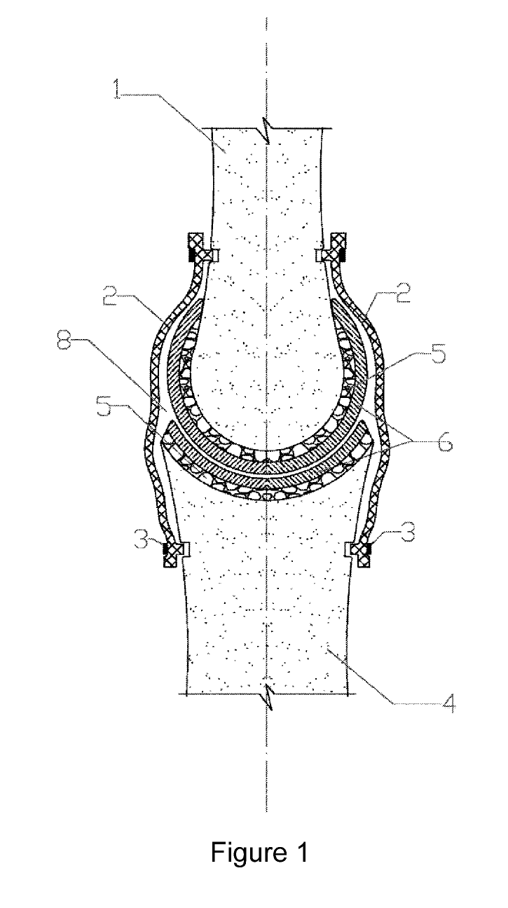

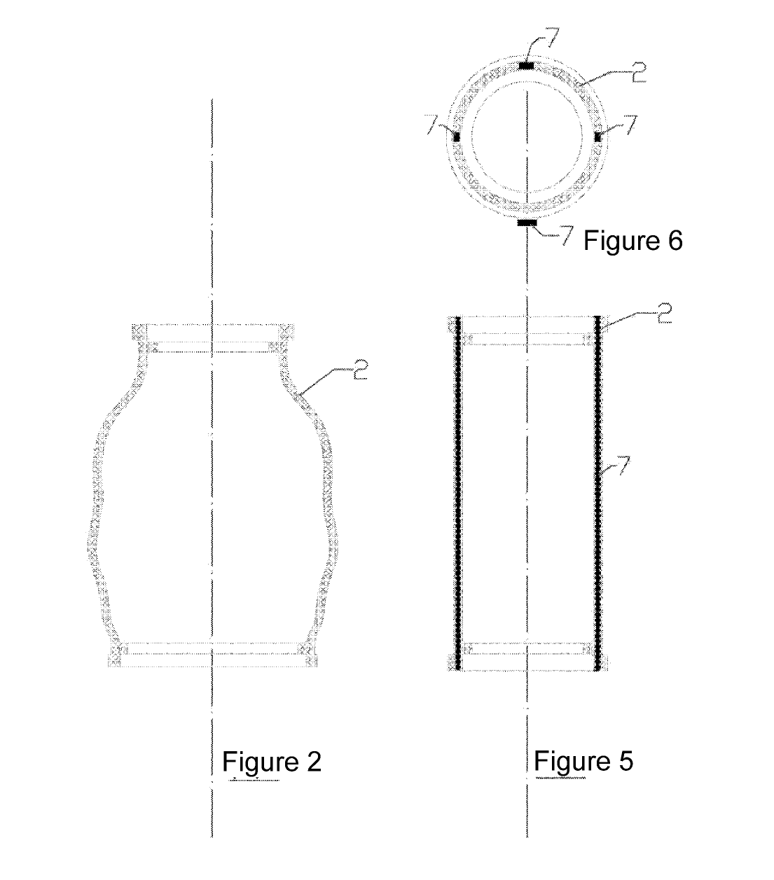

As shown in FIG. 1, the bionic joint device with closed lubrication comprises: a master bionic bone 1, a slave bionic bone 4, an elastic cavity housing 2 as indicated separately in FIG. 2, FIG. 5, and FIG. 6, a cavity sealing clamp 3, an elastic shock-absorbing material 5, a high-strength, wear-resistant, self-lubricating material 6, a powerful inelastic limit connection 7 that is indicated in FIG. 2, FIG. 5, and FIG. 6, and can either be placed inside the elastic cavity or be fixed outside the elastic cavity, wherein the bionic joint device with closed lubrication is filled with a lubricating grease 8 that satisfies the lubrication requirements of the high-strength, wear-resistant, self-lubricating material 6 and maintains the lubrication and friction action between the master bionic bone 1 and the slave bionic bone 4; the high-strength, wear-resistant, self-lubricating material 6 ensures good bending, twisting, and sliding effects; the elastic cavity housing 2 maintains the friction and lubrication action and prevents leakage of the lubricating grease contained in the elastic cavity housing 2 in the process of bending and stretching; the cavity sealing clamp 3 perfectly seals the elastic cavity housing 2 by means of the clamping slots of the master bionic bone 1 and the slave bionic bone 4; and glue is applied on the contact surfaces to prevent leakage of the lubricating grease 8 contained in the elastic cavity housing 2. The elastic shock-absorbing material 5 is used to mitigate external impacts and vibrations; the high-strength, wear-resistant, self-lubricating material 6 is used to reduce the friction force between the contact surfaces and to maintain the durability under friction; the powerful inelastic limit connection 7 can (i) control the angle of backward bending if it is placed beneath the cavity sealing clamp 3 in the front externally and is appropriately longer than the elastic cavity housing 2; (ii) control the angle of forward bending to the maximum of 180° if it is placed in the back and is as long as the elastic cavity housing 2; (iii) limit the gap size between the master bionic bone 1 and the slave bionic bone 4, prevent the dislocation between the master bionic bone 1 and the slave bionic bone 4 arising from the pulling force exerted by external force to the elastic cavity housing 2, prevent vertical displacement, and protect the fastening connection if it is placed on both sides along the circumference; (iv) control the angle of backward bending and provide safety protection if it is placed at an angle of 45° along the circumference and is as long as the elastic cavity housing 2, and (v) provide various limiting functions as needed if it is placed at different positions along the circumference.

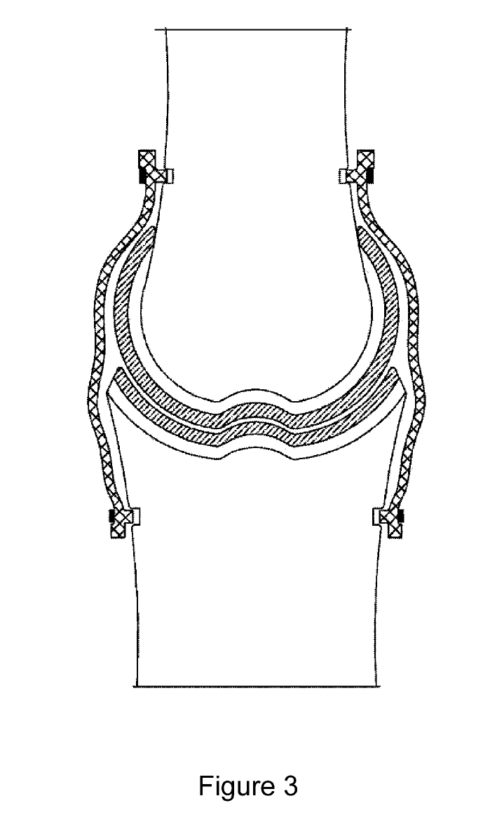

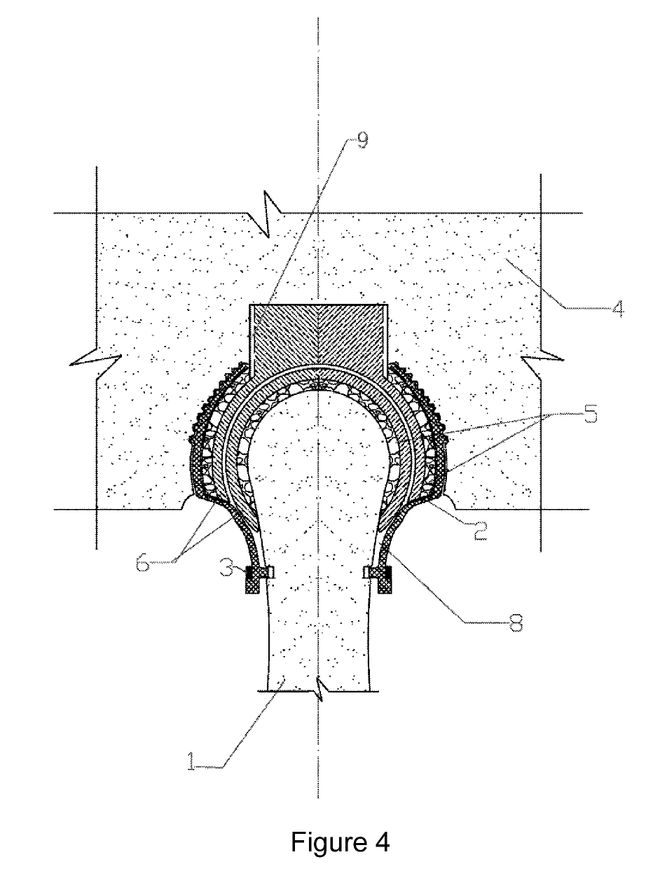

To sum up, both the direction and mode of motion of the joint can also be changed by changing the contact points on the master bionic bone 1, the slave bionic bone 4, and the contact surfaces. A spherical contact surface can rotate and act multi-directionally. FIG. 4 shows a sectional view of a spherical, rotatable hip joint provided by the present invention. The spherical, rotatable hip joint works on the same principle as shown in FIG. 1, and can rotate and act multi-directionally. The bionic joint is pressed by means of a threaded connection 9, thereby sealing the cavity perfectly. A bionic joint, if it has a 2-point contact surface, can bend and act only forwards and backwards. FIG. 3 shows a sectional view of a knee joint provided by the present invention, which has two contact points and can bend forwards and backwards. Meanwhile, a socket groove is available to support the joint. Other structures are similar. If a joint has a contact surface with three or more contact points, the joint for example, the spine joint of the human body can only bend and twist at a small angle. If varied in its size and shape, the bionic joint device with closed lubrication can be applied to all animal joints and humanoid robots.

Based on the foregoing mode of motion, the bionic joint device with closed lubrication can vary in size and shape as needed by the joints in different parts of the human body, in animals, and in humanoid robots, thereby enabling the joints to act more flexibly and freely. With the advances in the micromachine field, the bionic joint device with closed lubrication can also be applied to tiny animals. It is characterized in that soft and elastic materials are used to perfectly maintain motion and bone connection and mitigate various impacts and vibrations.

To sum up, the present invention integrates the achievements in the fields of bionics, machinery, and lubrication structure, and can be easily processed into a large-size product. The present invention also can be made into micro-sized joints by means of modern micromachining technology and can finally be applied in diversified fields. Such mechanical bionic joints can be attached to an articulated metal skeleton or framework of humanoid robots or quadruped machinery, enabling the humanoid robots or quadruped machinery to act more flexibly and freely. Meanwhile, such mechanical bionic joints can also be applied to a diversity of mechanical arms and mechanical hands.

The present invention enables a diversity of joint motions by means of the bionic joint device with closed lubrication, and is characterized by a simple structure, cost effectiveness, ease of production, and ease of replacement if damaged. The present invention also enables the bionic joint to bend and act by means of closed lubrication. Meanwhile, the inelastic position-limiting connection can control and limit the bending, prevent vertical displacements and falls, provide safety protection, and ensure the stability and safety of the joint. As an innovative joint device, the present invention can be applied to the machinery and robot fields on a large scale.

1. A bionic joint device with closed lubrication, comprising a master bionic bone, a slave bionic bone, an elastic cavity housing, a cavity sealing clamp, an elastic shock-absorbing material, a high-strength, wear-resistant, self-lubricating material, and a powerful inelastic positioning connection, characterized in that the elastic cavity housing is filled with a lubricating grease that satisfies the lubrication requirements of the high-strength, wear-resistant, self-lubricating material; the lubricating grease maintains the lubrication action between the master bionic bone and the slave bionic bone; the elastic cavity housing and the lubricating grease protect the high-strength, wear-resistant self-lubricating material and ensure a good bending, twisting, and sliding performance of the joint device; the elastic cavity housing maintains lubrication action and prevents leakage of lubricating grease in the process of bending and stretching; the cavity sealing clamp seals the elastic cavity housing by means of the clamping slots of the master bionic bone and the slave bionic bone; and glue is applied on the contact surfaces to prevent leakage of the lubricating grease contained inside the elastic cavity housing.

2. The bionic joint device with closed lubrication according to claim 1, further characterized in that the elastic shock-absorbing material is used to mitigate external impacts and vibrations; the high-strength, wear-resistant, self-lubricating material is used to reduce the friction force between the contact surfaces and prolong their useful life; the powerful inelastic limit connection can be placed at multiple positions along the circumference as needed according to the bending direction with a view to limiting the bending degree; and the powerful inelastic limit connection controls the gap size between the master bionic bone and the slave bionic bone.

3. The bionic joint device with closed lubrication according to claim 1, which can be used as a spherical, rotatable hip joint.
